(19) United States
(12) Patent Application Publication
(10) Pub. No.: US 2026/0315347 A1
(43) Pub. Date: Oct. 15, 2026

(54) BIOBASED ADDITIVE FOR PERSONAL CARE PRODUCTS

(71) Applicant: Solugen, Inc., Houston, TX (US)

(72) Inventors: Catherine Gonzalez, Houston, TX (US); Ju Su An, Houston, TX (US); Jason Helander, Richmond, TX (US); Chris Arceo, Richmond, TX (US); Amit Desai, Sugar Land, TX (US); Shauheen Noorani, Houston, TX (US)

(73) Assignee: Solugen, Inc., Houston, TX (US)

(21) Appl. No.: 19/484,978

(22) PCT Filed: May 30, 2024
(86) PCT No.: PCT/US2024/031799

§ 371 Date: Nov. 17, 2025

Related U.S. Application Data
(60) Provisional application No. 63/504,971, filed on May 30, 2023.

(51) Int. Cl. A61K 8/365; A61K 8/04; A61Q 1/00; A61Q 19/00; A61Q 19/10

(52) U.S. Cl. CPC A61K 8/365; A61K 8/042; A61Q 1/00; A61Q 19/007; A61Q 19/10; A61K 2800/30; A61K 2800/524

(57) ABSTRACT

An additive comprising (i) a sugar oxidation product and (ii) a solvent. A personal care product comprising (i) an additive comprising (1) a sugar oxidation product and (2) at least one material selected from the group consisting essentially of a solvent, chelating agent, emollient, humectant, preservative and combinations thereof and (ii) at least one material selected from the group consisting essentially of an emollient, a preservative, a paraben, formaldehyde, a fragrance, an aldehyde, glycerin, a humectant, an emulsifier, citric acid, benzophenone, oxybenzone, zinc oxide, titanium dioxide, a surfactant, sodium lauryl sulfate, toluene, acetone, a solvent, sodium bicarbonate colorants, thickeners, stabilizers, preservatives, pH adjusters, pH buffers, silicones, natural extracts, antioxidants, thereof co-surfactants, conditioning agents, anti-dandruff agents, anti-microbial agents, suspending agents, pearlescent aids, foam boosters, pediculocides, proteins, vitamins, amino acids, skin active agents, sunscreens, UV absorbers, stabilizers, and combinations thereof.

### CROSS-REFERENCE TO RELATED APPLICATIONS

This application is a 35 U.S.C. § 371 national stage application of PCT/US2024/031799 filed May 30, 2024 and entitled “Biobased Additive for Personal Care Products,” which is related to U.S. Provisional Application Ser. No. 63/504,971 filed May 30, 2023 entitled “BIOBASED ADDITIVE FOR PERSONAL CARE PRODUCTS,” each of which is hereby incorporated herein by reference in its entirety for all purposes.

### FIELD

The present disclosure relates generally to biobased additives. More particularly, the present disclosure relates to biobased additives for use in personal care products.

### BACKGROUND

Multifunctional, environmentally-friendly, and non-toxic products are in high demand. For personal care product manufacturers, there are two critical areas that will be impacted by rising consumer demand for the aforementioned characteristics: (i) recipe formulation and (ii) ingredient traceability. Many personal care brands are evolving their product composition to incorporate more natural and ethically-harvested ingredients. Additionally, personal care brands are increasingly focusing on authenticity in marketing their clean credentials.

Conventional additives used in personal care products, while effective, often (i) are not biodegradable, (ii) are biodegradable but utilize harmful additives, or (iii) are biodegradable but exhibit inadequate performance. For example, ethylenediaminetetraacetic acid, EDTA, a common chelating agent, is known to be a persistent pollutant in the environment. Common chelating agents are not biodegradable and tend to lead to the mobilization and bioavailability of heavy metals. Furthermore, for example, free EDTA is shown to produce adverse reproductive and developmental effects in mammals. Thus, an ongoing need exists for additives that improve the performance of personal care products.

### BRIEF DESCRIPTION OF THE FIGURES

The following FIGURE forms part of the present specification and is included to further demonstrate certain aspects of the present disclosure. The subject matter of the present disclosure may be better understood by reference to the FIGURE in combination with the detailed description of specific aspects presented herein.

FIG. 1 is a schematic depiction of gluconic and glucaric acid.

While the subject matter disclosed herein is susceptible to various modifications and alternative forms, only a few specific aspects have been shown by way of example in the drawing and are described below in detail. The figures and detailed descriptions of these specific aspects are not intended to limit the breadth or scope of the subject matter disclosed or the appended claims in any manner. Rather, the figures and detailed written descriptions are provided to illustrate the present disclosure to a person skilled in the art and to enable such person to make and use the concepts disclosed herein.

### SUMMARY

An additive comprising (i) a sugar oxidation product and (ii) a solvent.

A personal care product comprising (i) an additive comprising (1) a sugar oxidation product and (2) at least one material selected from the group consisting essentially of a solvent, chelating agent, emollient, humectant, preservative and combinations thereof and (ii) at least one material selected from the group consisting essentially of an emollient, a preservative, a paraben, formaldehyde, a fragrance, an aldehyde, glycerin, a humectant, an emulsifier, citric acid, benzophenone, oxybenzone, zinc oxide, titanium dioxide, a surfactant, sodium lauryl sulfate, toluene, acetone, a solvent, sodium bicarbonate colorants, thickeners, stabilizers, preservatives, pH adjusters, pH buffers, silicones, natural extracts, antioxidants, thereof co-surfactants, conditioning agents, anti-dandruff agents, anti-microbial agents, suspending agents, pearlescent aids, foam boosters, pediculocides, proteins, vitamins, amino acids, skin active agents, sunscreens, UV absorbers, stabilizers, and combinations thereof.

### DETAILED DESCRIPTION

Disclosed herein are biobased additives (BBA) for use in personal care and hygiene products. Herein the term “biobased” refers to the feedstocks or starting materials that are used to produce the material. Biobased additives of the type disclosed herein are composed in whole, or in considerable part, of biological products, renewable agricultural materials, or forestry materials. Biobased compounds are often an alternative to their petroleum-based counterparts. In an aspect, the BBA comprises at least one sugar oxidation product. Sugar oxidation products of the type disclosed herein may be characterized by at least one diacid functionality.

In an aspect, the BBA comprises one or more sugar oxidation products. In an aspect, the one or more sugar oxidation products comprise aldonic acid, uronic acid, aldaric acid, or a combination thereof and a counter cation. For example, the one or more sugar oxidation products may be a mixture of aldaric, uronic acids, and their respective counter-cations.

Additionally or alternatively, in an aspect, the one or more sugar oxidation products comprise a glucose oxidation product, a gluconic acid oxidation product, a gluconate, or combinations thereof. The glucose oxidation product, gluconic acid oxidation product, or combination thereof may be buffered to a suitable pH.

Additionally or alternatively, in one or more aspects, the one or more sugar oxidation products comprise glucaric acid, gluconic acid, glucuronic acid, glucose oxidation products, gluconic acid oxidation products, or combinations thereof. Additionally or alternatively, in one or more aspects, the one or more sugar oxidation products comprises disaccharides, oxidized disaccharides, uronic acid, aldaric acid or a combination thereof.

Additionally or alternatively, in one or more aspects, the one or more sugar oxidation products comprise gluconic acid, glucaric acid, glucuronic acid, n-keto-acids, C2 to C6 diacids, or combinations thereof.

Additionally or alternatively, in one or more aspects, the one or more sugar oxidation products comprise galactonic acid, galactaric acid, an oxidation product comprising predominantly (e.g., greater than about 50 weight percent) galactonic acid and/or galactaric acid with minor component species of n-keto-acids, C2 to C6 diacids, or combination thereof. Additionally or alternatively, in one or more aspects, the one or more sugar oxidation products comprise glutamic acid. Additionally or alternatively, in one or more aspects, the one or more sugar oxidation products comprise glucodialdose, 2-ketoglucose, or combinations thereof.

In such aspects, the buffered glucose oxidation product, the buffered gluconic acid oxidation product, or combinations thereof are buffered to a suitable pH. For example, the glucose oxidation product, gluconic acid oxidation product, or combination thereof may be buffered to a pH in the range of from about 1 to about 5. In various aspects, buffering of the one or more sugar oxidation products may be carried using any suitable acid, base, or combination thereof.

In one or more aspects, the one or more sugar oxidation products comprise aldonic acid, uronic acid, aldaric acid, a gluconic acid oxidation product, a gluconate, glucaric acid, gluconic acid, glucuronic acid, glucose oxidation products, galactonic acid, galactaric acid, glutamic acid, a lactone of gluconic acid, a lactone of glucaric acid, a lactone of galactaric acid, a lactone of galactonic acid, glucodialdose, 2-ketoglucose, disaccharides, oxidized disaccharides, n-keto-acids, C2 to C6 diacids, salts thereof, or combinations thereof.

In an aspect, the one or more sugar oxidation products comprise a glucose oxidation product, a gluconic acid oxidation product, a gluconate, glucaric acid, an oxidized glucuronolactone, a uronic acid oxidation product, or combinations thereof. Additionally or alternatively, the one or more sugar oxidation products comprises a buffered glucose oxidation product, a buffered gluconic acid oxidation product, or combinations thereof. In some such aspects, the buffered glucose oxidation product, the buffered gluconic acid oxidation product, or combinations thereof are buffered to a pH within a range disclosed herein with any suitable acid or base such as sodium hydroxide. In an example of such aspects, the one or more sugar oxidation product comprises a mixture of gluconic acid and glucaric acid and further comprises a minor component species comprising n-keto-acids, C2-C6 diacids or combinations thereof.

In an aspect, the one or more sugar oxidation products comprise glucodiamine, glucodialdose, 2-ketoglucose, glucaric acid, lactones of glucaric acid, gluconic acid, lactones of gluconic acid, galactonic acid, lactones of galactonic acid, galactaric acid, lactones of galactaric acid, galactonic acid glucoheptonic acid, lactones of glucoheptonic acid, or combinations thereof.

In one or more aspects, any of the one or more sugar oxidation products or combination of one or more sugar oxidation products disclosed herein may further comprise a counter-cation such as a Group 1 alkali metal, a Group 2 alkaline earth metal, a Group 8 metal, Group 11 metal, Group 12 metal, or combination thereof. For example, the counter-cation may comprise silicates, borates, aluminum, calcium, magnesium, ammonium, sodium, potassium, cesium, strontium, zinc, copper, ferric iron or ferrous iron, or combinations thereof.

In an aspect, the one or more one or more sugar oxidation products suitable for use in the present disclosure comprises less than about 5 wt. % maltose, maltotriose, fructose, higher molecular weight polysaccharides, oxidation products thereof, or combinations thereof based on the total weight of the sugar oxidation product. In one or more aspects, the one or more sugar oxidation products comprise aldonic acid, uronic acid, aldaric acid, or combination thereof.

In an aspect, the BBA comprises glucaric acid. In such aspects, glucaric acid may be present in the BBA in an amount ranging from about 1 weight percent (wt. %) to about 100 wt. %, additionally or alternatively from about 10 wt. %, alternatively, additionally to about 80 wt. % to about additionally or alternatively from about 0.1 wt. % to about 2 wt. %, additionally or alternatively from about 0.5 wt. % to about 2 wt. %, additionally or alternatively from about 1 wt. % to about 5 wt. %, additionally or alternatively from about 1 wt. % to about 25 wt. %, or additionally or alternatively from about 1 wt. % to about 10 wt. % based on the total weight of the BBA.

In an aspect, the BBA comprises gluconic acid. In such aspects, gluconic acid may be present in the BBA in an amount ranging from about 1 wt. % to about 100 wt. %, additionally or alternatively from about 10 wt. %, alternatively, additionally to about 80 wt. % to about additionally or alternatively from about 0.1 wt. % to about 2 wt. %, additionally or alternatively from about 0.5 wt. % to about 2 wt. %, additionally or alternatively from about 1 wt. % to about 5 wt. %, additionally or alternatively from about 1 wt. % to about 25 wt. %, or additionally or alternatively from about 1 wt. % to about 10 wt. % based on the total weight of the BBA.

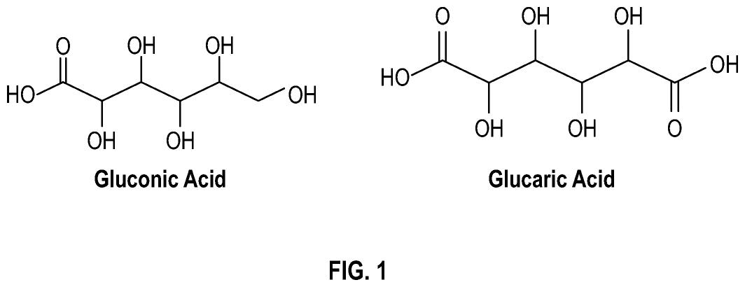

In an aspect, the BBA comprises a mixture of gluconic acid and glucaric acid. In such aspects, the ratio of gluconic acid:glucaric acid may be about 5:1, additionally or alternatively about 1:5, additionally or alternatively from about 3:1 to about 1:3 or, additionally or alternatively, from about 2:1 to about 1:2. In one or more aspects, the BBA has the mixture of gluconic acid present in an amount ranging from about 1 wt. % to about 100 wt. %, additionally or alternatively from about 10 wt. %, alternatively, additionally to about 80 wt. % to about additionally or alternatively from about 0.1 wt. % to about 2 wt. %, additionally or alternatively from about 0.5 wt. % to about 2 wt. %, additionally or alternatively from about 1 wt. % to about 5 wt. %, additionally or alternatively from about 1 wt. % to about 25 wt. %, or additionally or alternatively from about 1 wt. % to about 10 wt. % based on the total weight of the BBA. FIG. 1 depicts the chemical structures of gluconic acid and glucaric acid.

In one or more aspects, the BBA comprises an ALTIV biobased detergent builder commercially available from Solugen Inc.

In one or more aspects, the BBA further comprises a chelating agent. Herein a chelating agent refers to a compound able to form two or more separate coordinate bonds with a single central metal atom. Any suitable chelating agent may be present in the BBA in amounts effective to improve one or more performance characteristics of the BBA. Nonlimiting examples of chelating agents suitable for use in the present disclosure include gluconic acid, glucaric acid, galactonic acid, galactaric acid, glucoheptonic acid, trisodium phosphate, phosphonates, ethylenediaminetetraacetic acid (EDTA), nitrilotriacetic acid (NTA), methylglycinediacetic acid (MGDA), glutamic acid, N,N-diacetic acid (GLDA), ethylenediamine-N,N′-disuccinic acid (EDDS), diethylenetriaminepentaacetic acid (DTPA), hydroxyethylethylenediaminetriacetic acid (HEDTA), ethanoldiglycine (EDG), salts thereof, or combinations thereof.

The chelating agent may be present in the BBA in an amount ranging from about 0.1 wt. % to about 80 wt. %, additionally or alternatively, from about 0.1 wt. % to about 50 wt. %, additionally or alternatively from about 0.1 wt. % to about 10 wt. %, additionally or alternatively from about 0.1 wt. % to about 5 wt. %, additionally or alternatively from about 5 wt. % to about 30 wt. % based on the total weight of the BBA.

In some aspects, the BBA further comprises a solvent. In such aspects, the BBA includes an aqueous solvent in amounts sufficient to provide one or more user and/or process desired characteristics.

In one or more aspects, a BBA of the type disclosed herein is included in a personal care product. Herein personal care products refer to materials which are applied on various external parts of the body such as skin, hair, nails, lips, external genital and anal areas, as well as teeth and mucous membrane of the oral cavity, in order to, among other functions, make them clean, protect them from harmful germs and keep them in good condition. In one or more aspects, a BBA when a component of a personal care product may function to (i) improve skin texture, tone, and hydration; (ii) provide antioxidant benefits; (iii) act as an exfoliator (iv) stabilize the product; (iv) regulate pH, (v) act as a humectant, (vi) coordinate metal ions or (vii) protect oils and actives in skin care; (vii) protect products from discoloration (ix) protect products from degradation; (x) protect products from rancidity or (xi) combinations of (i) through (x).

In one or more aspects, a BBA suitable for use in the present disclosure may be included in a formulation comprising an emollient, a preservative, a paraben, formaldehyde, a fragrance, an aldehyde, glycerin, a humectant, an emulsifier, citric acid, benzophenone, oxybenzone, zinc oxide, titanium dioxide, a surfactant, sodium lauryl sulfate, toluene, acetone, a solvent, sodium bicarbonate colorants, thickeners, stabilizers, preservatives, pH adjusters, pH buffers, silicones, natural extracts, antioxidants, thereof co-surfactants, conditioning agents, anti-dandruff agents, anti-microbial agents, suspending agents, pearlescent aids, foam boosters, pediculocides, proteins, vitamins, amino acids, skin active agents, sunscreens, UV absorbers, stabilizers, and combinations thereof.

Nonlimiting examples of personal care and/or hygiene products include cleansing products, hair care products, oral care products, sun care products, skin hydrating products, feminine care products, hair removal products, nail care products, eye care products and anal hygiene products, odoriferous compounds such as perfumes, fragrances or aromas, hand soaps or bar soaps, shower gels, body washes, facial cleansers, body oils, body lotions, cleansing pads, moist towelettes, exfoliating scrubs, shampoos, hair conditioners, hair oils, hair treatments, scalp serums, shaving creams, shaving gels, shaving foams, depilatory creams, toothpaste, mouthwash, gum gel, powders, baby powders, body lotions, hand creams, pomades, facial moisturizers, sunscreen, nail care products, artificial tears, lubricating eye drops contact lens solutions; eye creams, eye, eyelid cleansers, eye masks anti-cellulite products, anti-wrinkle products, anti-dandruff products, hair straighteners, detanglers, reaffirmers, moisturizers, humectants, makeup-removers, pigment-removers, pearlizers, deodorizers, antiperspirants, whitening and bleaching substances, mouthwashes, bath products, cleaning products, skin care products, shampoos, rosacea treatments or combinations thereof.

A BBA may be included in a personal care product in any amount sufficient to meet one or more user and/or process goals. For example, the BBA may be present in the personal care product in amounts ranging from about 0.01 wt. % to about 20 wt. %, additionally or alternatively, from about 0.1 wt. % to about 10 wt. % or, additionally or alternatively, from about 0.1 wt. % to about 5 wt. % based on the total weight of the personal care product.

In one or more aspects, the BBA may function as a replacement for a gluconate salt that is present in a personal care product. In such aspects, the personal care product comprises less than about 0.5 weight percent (wt. %), alternatively less than about 0.25 wt. % or alternatively less than about 0.1 wt. % of a gluconate salt based on the total weight of the personal care product. In some aspects, the BBA is used in conjunction with a gluconate salt such that the amount of gluconate salt used is from about 10% to about 95% less than the amount used in the absence of the BBA. Alternatively, the gluconate salt may be used in conjunction with a BBA in amounts that range from about 5% to about 50%, alternatively form about 10% to about 40%, alternatively from about 25% to about 35%, alternatively from about 20% to about 80% or alternatively from about 25% to about 50% less than the amount used in the absence of a BBA.

In some aspects, a BBA is used in lieu of an alphahydroxy acid (AHA). In some aspects, the BBA is used in conjunction with an AHA such that the amount of AHA used is from about 10% to about 95% less than the amount used in the absence of the BBA. Alternatively, the AHA may be used in conjunction with a BBA in amounts that range from about 5% to about 50%, alternatively form about 10% to about 40%, alternatively from about 25% to about 35%, alternatively from about 20% to about 80% or alternatively from about 25% to about 50% less than the amount used in the absence of a BBA.

In one or more aspects, the BBA is used in personal care formulations as a preservative or as a preservative enhancer. In an aspect, the BBA is used as a preservative in the absence of any other preservative. In alternative aspects, the BBA is used in conjunction with a preservative such as formaldehyde, 1,3-bis(hydroxymethyl)-5,5-dimethylimidazolidine-2,4-dione, methenamine 3-chloroallylochloride, imidazolidinyl urea, diazolidinyl urea, polyoxymethylene urea, sodium hydroxymethylglycinate, 2-bromo-2-nitropropane-1,3-diol (bromopol), glyoxal, parabens, benzylate, benzoic acid, benzyl ester and combinations thereof. In such aspects, the BBA is used in conjunction with a preservative such that the amount of preservative used is from about 10% to about 95% less than the amount used in the absence of the BBA. Alternatively, the preservative may be used in conjunction with a BBA in amounts that range from about 5% to about 50%, alternatively form about 10% to about 40%, alternatively from about 25% to about 35%, alternatively from about 20% to about 80% or alternatively from about 25% to about 50% less than the amount used in the absence of a BBA.

In one or more aspects, the BBA is used in lieu of a chelating agent which functions to aid in preservation of formulations. In the alternative, the personal care product comprises a BBA and a chelating agent. Herein a chelating agent refers to a compound able to form two or more separate coordinate bonds with a single central metal atom. Nonlimiting examples of chelating agents include galactonic acid, galactaric acid, glucoheptonic acid, trisodium phosphate, phosphonates, ethylenediaminetetraacetic acid (EDTA), nitrilotriacetic acid (NTA), methylglycinediacetic acid (MGDA), glutamic acid, N,N-diacetic acid (GLDA), ethylenediamine-N,N′-disuccinic acid (EDDS), diethylenetriaminepentaacetic acid (DTPA), hydroxyethylethylenediaminetriacetic acid (HEDTA), ethanoldiglycine (EDG), salts thereof, or combinations thereof. In aspects, the where the BBA is used in conjunction with a chelating agent the amount of chelating agent used is from about 10% to about 95% less than the amount used in the absence of the BBA. Alternatively, the chelating agent may be used in conjunction with a BBA in amounts that range from about 5% to about 50%, alternatively form about 10% to about 40%, alternatively from about 25% to about 35%, alternatively from about 20% to about 80% or alternatively from about 25% to about 50% less than the amount used in the absence of a BBA.

In one or more aspects, the BBA is combined with a thickener to provide a rheology modifier. Nonlimiting examples of thickeners that may be used to form a VMA include xanthan, chitosan, guar, hydroxyethylcellulose, hyaluronic acid, cellulose, gelatin, silicates, carbomer thickeners and combinations thereof.

In one or more aspects, the BBA is combined with chitosan to form a gel like material which can function as a rheology modifier. Chitosan is a linear polysaccharide composed of randomly distributed β-linked D-glucosamine and N-acetyl-D-glucosamine.

In another aspect, the BBA when combined with chitosan functions as a cationic emulsifier. This new generation of cationic emulsifiers are being introduced as new technologies that are mild to the skin and come with a better eco/sustainability stance than before. Anionic polymer stabilizers are not appropriate for the task and nonionic polymer stabilizers often do not produce rheological or tactile properties that are appealing. Rheology modifiers comprising for example a BBA and a thickener (e.g., chitosan) can provide a differentiated aesthetic, film forming, manufacturing ease, skin smoothing and preservation enhancement.

In one or more aspects, the BBA functions as a pH adjuster in a cleansing product.

In one or more aspects, the BBA functions as an exfoliator, moisturizer or both in a skincare personal product.

### ADDITIONAL DISCLOSURE

The following are nonlimiting exemplary aspects of the presently disclosed subject matter.

A first aspect which is an additive comprising (i) a sugar oxidation product and (ii) a solvent.

A second aspect which is the additive of the first aspect wherein the sugar oxidation product comprises gluconic acid in an amount of from about 1 wt. % to about 100 wt. % based on the total weight of the additive.

A third aspect which is the additive of any of the first through second aspects wherein the sugar oxidation product comprises glucaric acid in an amount of from about 1 wt. % to about 100 wt. % based on the total weight of the additive.

A fourth aspect which is the additive of any of the first through third aspects wherein the sugar oxidation product comprises a mixture of gluconic acid and glucaric acid having a ratio of gluconic acid:glucaric acid of about 5:1.

A fifth aspect which is the additive of any of the first through fourth aspects comprising a chelating agent.

A sixth aspect which is the additive of the fifth aspect wherein the chelating agent comprises galactonic acid, galactaric acid, glucoheptonic acid, trisodium phosphate, phosphonates, ethylenediaminetetraacetic acid (EDTA), nitrilotriacetic acid (NTA), methylglycinediacetic acid (MGDA), glutamic acid, N,N-diacetic acid (GLDA), ethylenediamine-N,N′-disuccinic acid (EDDS), diethylenetriaminepentaacetic acid (DTPA), hydroxyethylethylenediaminetriacetic acid (HEDTA), ethanoldiglycine (EDG), salts thereof or combinations thereof.

A seventh aspect which is the additive of any of the fifth through sixth aspects wherein the chelating agent is present in an amount of from about 0.1 wt. % to about 80 wt. % based on the total weight of the additive.

An eighth aspect which is the additive of any of the first through seventh aspects comprising a solvent.

A ninth aspect which is the additive of the eight aspect wherein the solvent comprises an aqueous fluid.

A tenth aspect which is a personal care product comprising (i) an additive comprising (1) a sugar oxidation product and (2) at least one material selected from the group consisting essentially of a solvent, chelating agent, emollient, humectant, preservative and combinations thereof and (ii) at least one material selected from the group consisting essentially of an emollient, a preservative, a paraben, formaldehyde, a fragrance, an aldehyde, glycerin, a humectant, an emulsifier, citric acid, benzophenone, oxybenzone, zinc oxide, titanium dioxide, a surfactant, sodium lauryl sulfate, toluene, acetone, a solvent, sodium bicarbonate colorants, thickeners, stabilizers, preservatives, pH adjusters, pH buffers, silicones, natural extracts, antioxidants, thereof co-surfactants, conditioning agents, anti-dandruff agents, anti-microbial agents, suspending agents, pearlescent aids, foam boosters, pediculocides, proteins, vitamins, amino acids, skin active agents, sunscreens, UV absorbers, stabilizers, and combinations thereof.

An eleventh aspect which is the product of the tenth aspect wherein the additive comprises at least two materials selected from the group consisting essentially of an emollient, a preservative, a paraben, formaldehyde, a fragrance, an aldehyde, glycerin, a humectant, an emulsifier, citric acid, benzophenone, oxybenzone, zinc oxide, titanium dioxide, a surfactant, sodium lauryl sulfate, toluene, acetone, a solvent, sodium bicarbonate colorants, thickeners, stabilizers, preservatives, pH adjusters, pH buffers, silicones, natural extracts, antioxidants, thereof co-surfactants, conditioning agents, anti-dandruff agents, anti-microbial agents, suspending agents, pearlescent aids, foam boosters, pediculocides, proteins, vitamins, amino acids, skin active agents, sunscreens, UV absorbers, stabilizers, and combinations thereof.

A twelfth aspect which is the product of any of the tenth through eleventh aspects wherein the additive comprises at least two materials

A thirteenth aspect which is the product of any of the tenth through twelfth aspects wherein the sugar oxidation product comprises a mixture of gluconic acid and glucaric acid having a ratio of gluconic acid:glucaric acid of about 5:1.

A fourteenth aspect which is the product of any of the tenth through thirteenth aspects comprising a chelating agent.

A fifteenth aspect which is the product of the fourteenth aspect wherein the chelating agent comprises galactonic acid, galactaric acid, glucoheptonic acid, trisodium phosphate, phosphonates, ethylenediaminetetraacetic acid (EDTA), nitrilotriacetic acid (NTA), methylglycinediacetic acid (MGDA), glutamic acid, N,N-diacetic acid (GLDA), ethylenediamine-N,N′-disuccinic acid (EDDS), diethylenetriaminepentaacetic acid (DTPA), hydroxyethylethylenediaminetriacetic acid (HEDTA), ethanoldiglycine (EDG), salts thereof or combinations thereof.

A sixteenth aspect which is the product of any of the tenth through fifteenth aspects comprising a solvent.

A seventeenth aspect which is the product of the sixteenth aspect wherein the solvent comprises an aqueous fluid.

An eighteenth aspect which is the product of any of the tenth through seventeenth aspects wherein the sugar oxidation product is present in an amount ranging from about 1 wt. % to about 100 wt. %.

A nineteenth aspect which is the product of any of the tenth through eighteenth aspects comprising a preservative.

A twentieth aspect which is the product of the nineteenth aspect wherein the preservative comprises formaldehyde, 1,3-bis(hydroxymethyl)-5,5-dimethylimidazolidine-2,4-dione, methenamine 3-chloroallylochloride, imidazolidinyl urea, diazolidinyl urea, polyoxymethylene urea, sodium hydroxymethylglycinate, 2-bromo-2-nitropropane-1,3-diol (bromopol), glyoxal, parabens, benzylate, benzoic acid, benzyl ester and combinations thereof.

### EXAMPLES

The presently disclosed subject matter having been generally described, the following examples are given as particular aspects of the subject matter and to demonstrate the practice and advantages thereof. It is understood that the examples are given by way of illustration and are not intended to limit the specification or the claims in any manner.

### Example 1

The utilization of a BBA of the type disclosed herein in a gel moisturizer formulation was investigated. An oil-free polyhydroxy acid (PHA) gel moisturizer was formulated that contained a BBA, specifically a sugar oxidation product. The PHA gel moisturizer was prepared by blending individual components of the gel moisturizer separately to form mixtures that were then incorporated into the final formulation. The individual components were blended to form Phase A which contained the BBA and water, Phase B which contained propanediol and xanthan gum and Phase C which contained silica.

The final formulation was prepared by premixing phase B and then adding the premixed phase B to phase A with agitation (e.g., fast propeller stirring) until the mixture was uniform. Phase C was then added to the uniform mixture with agitation until the mixture was uniform. This mixture was designated Formulation 1. The cosmetic benefits of Formulation 1 include but are not limited to: (i) line blurring and aesthetic enhancement; (ii) exfoliation and moisturization activity and (iii) a smoothing effect such that the formulation can act as a makeup primer. The component list is presented in Table 1.

TABLE 1

| Phase | % | COMPOUND | Function |
|---|---|---|---|
| A | 20 | BBA | Acid |
| A | 52 | Water | Solvent |
| B | 25 | Propanediol | Humectant |
| B | 1 | Xanthan Gum | Active |
| C | 2 | Silica | Thickener |

### Example 2

The utilization of a BBA of the type disclosed herein in a cream formulation was investigated. Specifically, a brown sugar PHA cream was formulated by creating 3 phases. Phase A contained water and a BBA, Phase B contained a triglyceride, an alcohol and a sulfosuccinate; and Phase C contained propanediol, silica, a phosphate, an acid, glycerol, a surfactant, and natural extracts. This formulation was designated Formulation 2, and the specific amount of these components listed in Table 2.

TABLE 2

| Phase | % | COMPOUND | Function |
|---|---|---|---|
| A | 53 | Water | Solvent |
| A | 10 | Gluconic acid | Acid |
| B | 15 | Caprylic/Capric Triglyceride | Emollient |
| B | 2.5 | Cetearyl alcohol | Emulsifier |
| B | 2.5 | Disodium Cetearyl Sulfosuccinate | Emulsifier |
| C | 10 | Propanediol | Humectant |
| C | 2.5 | Hydroxypropyl Starch Phosphate | Thickener |
| C | 2 | Silica | Aesthetic Modifier |
| C | 1 | Caprylhydroxamic Acid (and) | Preservative |
| | | Caprylyl Glycol (and) Glycerin | |
| C | 1 | Maltol (and) Polyquaternium-80 | Preservative |
| C | 0.5 | Camellia Sinensis Leaf Extract | Antioxidant |
| | | (and) Rosmarinus Officinalis | |
| | | (Rosemary) Leaf Extract (and) | |
| | | Malpighia Glabra (Acerola) | |
| | | Fruit Extract | |

Formulation 2 was prepared by heating phases A and B to 75° C.; phase B was added to phase A with agitation (e.g., fast sharp-bladed stirring) to form a mixture. Phase C, which was premixed, was added to the mixture and homogenized until uniform to generate Formulation 2.

### Example 3

The utilization of a BBA of the type disclosed herein in a facial cleanser was investigated. Specifically, a brown sugar facial cleanser was formulated by creating 3 phases. Phase A contained water and a BBA, Phase B contained water, a sulfonate, an alcohol and a sulfosuccinate; and Phase C contained propanediol, silica, a betaine and a stearamide and Phase C contained sodium chloride. This formulation was designated Formulation 3, and the specific amount of these components listed in Table 3.

TABLE 3

| Phase | % | COMPOUND | Function |
|---|---|---|---|
| A | 25.5 | Water | Solvent |
| A | 20 | Gluconic Acid | Acid |
| A | 5 | Propanediol | Humectant |
| B | 30 | Water (and) Sodium C14-16 Olefin Sulfonate | Surfactant |
| B | 15 | Water (and) Cocamidopropyl Betaine | Surfactant |
| B | 3 | PPG-2 Hydroxyethyl Coco/Isostearamide | Surfactant |
| C | 1.5 | Sodium Chloride | Thickener |

Formulation 3 was prepared by premixing phase A and phase B which was subsequently stirred, by hand or using a round propeller to form a mixture. Phase C was slowly added to the mixture until the desired viscosity was reached. Formulation 3 functioned as a gentle face cleanser.

### Example 4

The utilization of a BBA of the type disclosed herein in a soap formulation was investigated. The specific components and the amount of each component are present in Table 4. The table presents the amount of each component relative to the amount of the active which is set as 100%. BBA 1 contained a single sugar oxidation product while BBA2 contained a mixture of gluconic and glucaric acids.

TABLE 4

| | % formula as | | | | | | | |
|---|---|---|---|---|---|---|---|---|
| ingredient | 100% active | | | | | | | |
| Water | to 100 | to 100 | to 100 | to 100 | to 100 | to 100 | to 100 | to 100 |
| Cocoamidopropyl Hydroxysultaine | 4 | 4 | | | | | 4 | 4 |
| Sodium Methyl 2-Sulfolaurate | 2 | 2 | 2 | 2 | 2 | 2 | | |
| Glycerin | 2 | 2 | 2 | 2 | 2 | 2 | 2 | 2 |
| Olea Europea (Olive) Fruit Oil | 0.5 | 0.5 | 0.5 | 0.5 | 0.5 | 0.5 | 0.5 | 0.5 |
| Aloe Barbadensis Leaf Juice | 0.5 | 0.5 | 0.5 | 0.5 | 0.5 | 0.5 | 0.5 | 0.5 |
| Sodium chloride** | 1 | 1 | 1 | 1 | 1 | 1 | 1 | 1 |
| Potassium sorbate | 0.75 | 0.75 | 0.75 | 0.75 | 0.75 | 0.75 | 0.75 | 0.75 |
| Sodium benzoate | 0.75 | 0.75 | 0.75 | 0.75 | 0.75 | 0.75 | 0.75 | 0.75 |
| BBA 1 | 0.5 | | 0.5 | | 0.5 | | 0.5 | |
| BBA 2 | | 0.5 | | 0.5 | | 0.5 | | 0.5 |
| Caproyl/Capryloyl Methyl Glucamide | | | 4 | 4 | | | | |
| Lauroyl (C12/14) methyl glucamide | | | | | 4 | 4 | | |
| Oleic based sophorolipid (acidic form) | | | | | | | 2 | 2 |

The soap was prepared adding water to the surfactants (e.g., hydroxysultaine) and mixing to form a surfactant solution. Glycerin and extracts (e.g., fruit oil) was then added to the surfactant solution and agitated to form a mixture. Acids, sorbate and benozate was added to the mixture to form a second mixture. The pH of the second mixture as adjusted by addition of an acid (e.g., hydrochloric acid) to second mixture to adjust the pH to approximately 5.0. Sodium chloride was then added to the pH adjusted second mixture to form a soap.

### Example 3

Experiment: Preservation Study with BBA

The ability of a BBA of the type disclosed herein to function as a preservative was investigated. Specifically, a BBA was used as an alternative to EDTA in order to sequester cations and preserve formulations such as soaps. Each sample contained sodium tallowate, sodium cocoate, or sodium palm kernelate, water, sodium chloride, sodium silicate and magnesium sulfate and either no additive (control), EDTA, a mixture of glucaric acid and gluconic acid (GOGA) or gluconic acid (LG60). The samples were aged at 45° C. for eight weeks and the samples assessed weekly.

As shown in Table 5 below, the formulation containing a BBA (e.g., GOGA) was comparable to EDTA's preservation performance. The dosage of each chelate was 0.4% active product.

TABLE 5

| | Initial | 1 week | 2 weeks | 3 weeks | 4 weeks | 5 weeks | 8 weeks |
|---|---|---|---|---|---|---|---|
| control | Solid white, | Solid white, | Solid white, | Solid white, | Solid white, still | Solid white, this | Solid white, this |
| | no apparent | maybe | slightly souring | slightly sour when | smells slightly | one is still the | one is still the |
| | smell | starting to | | compared to | sour when | most sour | most sour |
| | | sour just a | | GOGA and EDTA | compared to | smelling | smelling |
| | | little | | | GOGA and EDTA | | |
| EDTA | Off white | Off white, no | Off white, no | Off white, no | Off white, still | Off white, still | Off white, still |
| | no apparent | apparent | apparent smell | apparent smell | smells fine | smells fine | smells fine |
| | smell | smell | | | | | |
| GOGA | Off white, | Off white, no | Off white, no | Off white, no | Off white, still | Off white, still | Off white, still |
| | no apparent | apparent | apparent smell | apparent smell | smells fine | smells fine | smells fine |
| | smell | smell | | | | | |
| LG60 | Tan, no | Tan, no | Tan, no | Tan, maybe | Tan, definitely | Tan, not much | Tan, slightly |
| | apparent | apparent | apparent smell | starting to sour | starting to sour | different than last | sour when |
| | smell | smell | | just a little when | when compared to | week | compared to |
| | | | | compared to | GOGA and EDTA | | GOGA and |
| | | | | GOGA and EDTA | | | EDTA |

While aspects of the presently disclosed subject matter have been shown and described, modifications thereof can be made by one skilled in the art without departing from the spirit and teachings of the subject matter. The aspects described herein are exemplary only and are not intended to be limiting. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the presently disclosed subject matter. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. An additive comprising (i) a sugar oxidation product and (ii) a solvent.

2. The additive of claim 1, wherein the sugar oxidation product comprises gluconic acid in an amount of from about 1 wt. % to about 100 wt. % based on the total weight of the additive.

3. The additive of claim 1, wherein the sugar oxidation product comprises glucaric acid in an amount of from about 1 wt. % to about 100 wt. % based on the total weight of the additive.

4. The additive of claim 1, wherein the sugar oxidation product comprises a mixture of gluconic acid and glucaric acid having a ratio of gluconic acid:glucaric acid of about 5:1.

5. The additive of claim 1, comprising a chelating agent.

6. The additive of claim 5, wherein the chelating agent comprises galactonic acid, galactaric acid, glucoheptonic acid, trisodium phosphate, phosphonates, ethylenediaminetetraacetic acid (EDTA), nitrilotriacetic acid (NTA), methylglycinediacetic acid (MGDA), glutamic acid, N,N-diacetic acid (GLDA), ethylenediamine-N,N′-disuccinic acid (EDDS), diethylenetriaminepentaacetic acid (DTPA), hydroxyethylethylenediaminetriacetic acid (HEDTA), ethanoldiglycine (EDG), salts thereof or combinations thereof.

7. The additive of claim 5, wherein the chelating agent is present in an amount of from about 0.1 wt. % to about 80 wt. % based on the total weight of the additive.

8. The additive of claim 1, comprising a solvent.

9. The additive of claim 8, wherein the solvent comprises an aqueous fluid.

10. A personal care product comprising (i) an additive comprising (1) a sugar oxidation product and (2) at least one material selected from the group consisting essentially of a solvent, chelating agent, emollient, humectant, preservative and combinations thereof and (ii) at least one material selected from the group consisting essentially of an emollient, a preservative, a paraben, formaldehyde, a fragrance, an aldehyde, glycerin, a humectant, an emulsifier, citric acid, benzophenone, oxybenzone, zinc oxide, titanium dioxide, a surfactant, sodium lauryl sulfate, toluene, acetone, a solvent, sodium bicarbonate colorants, thickeners, stabilizers, preservatives, pH adjusters, pH buffers, silicones, natural extracts, antioxidants, thereof co-surfactants, conditioning agents, anti-dandruff agents, anti-microbial agents, suspending agents, pearlescent aids, foam boosters, pediculocides, proteins, vitamins, amino acids, skin active agents, sunscreens, UV absorbers, stabilizers, and combinations thereof.

11. The product of claim 10, wherein the additive comprises at least two materials selected from the group consisting essentially of an emollient, a preservative, a paraben, formaldehyde, a fragrance, an aldehyde, glycerin, a humectant, an emulsifier, citric acid, benzophenone, oxybenzone, zinc oxide, titanium dioxide, a surfactant, sodium lauryl sulfate, toluene, acetone, a solvent, sodium bicarbonate colorants, thickeners, stabilizers, preservatives, pH adjusters, pH buffers, silicones, natural extracts, antioxidants, thereof co-surfactants, conditioning agents, anti-dandruff agents, anti-microbial agents, suspending agents, pearlescent aids, foam boosters, pediculocides, proteins, vitamins, amino acids, skin active agents, sunscreens, UV absorbers, stabilizers, and combinations thereof.

12. The product of claim 10, wherein the additive comprises at least two materials.

13. The product of claim 10, wherein the sugar oxidation product comprises a mixture of gluconic acid and glucaric acid having a ratio of gluconic acid:glucaric acid of about 5:1.

14. The product of claim 10, comprising a chelating agent.

15. The product of claim 14, wherein the chelating agent comprises galactonic acid, galactaric acid, glucoheptonic acid, trisodium phosphate, phosphonates, ethylenediaminetetraacetic acid (EDTA), nitrilotriacetic acid (NTA), methylglycinediacetic acid (MGDA), glutamic acid, N,N-diacetic acid (GLDA), ethylenediamine-N,N′-disuccinic acid (EDDS), diethylenetriaminepentaacetic acid (DTPA), hydroxyethylethylenediaminetriacetic acid (HEDTA), ethanoldiglycine (EDG), salts thereof or combinations thereof.

16. The product of claim 10, comprising a solvent.

17. The product of claim 16, wherein the solvent comprises an aqueous fluid.

18. The product of claim 10, wherein the sugar oxidation product is present in an amount ranging from about 1 wt. % to about 100 wt. %.

19. The product of claim 10, comprising a preservative.

20. The product of claim 19, wherein the preservative comprises formaldehyde, 1,3-bis(hydroxymethyl)-5,5-dimethylimidazolidine-2,4-dione, methenamine 3-chloroallylochloride, imidazolidinyl urea, diazolidinyl urea, polyoxymethylene urea, sodium hydroxymethylglycinate, 2-bromo-2-nitropropane-1,3-diol (bromopol), glyoxal, parabens, benzylate, benzoic acid, benzyl ester and combinations thereof.
